(19) United States
(12) Patent Application Publication
(10) Pub. No.: US 2008/0177289 A1
(43) Pub. Date: Jul. 24, 2008

(54) Device For Sewing Elements That Can Be Sutured

(76) Inventors: Carlo Angelo Adami, Negrar (IT); Roberto Calvi, Montevecchia (IT); Luciano Patorno, Saliceto San Giuliano (IT); Giuseppe Tuscano, Saliceto San Giuliano (IT)

Correspondence Address: Modiano & Associati, Via Meravigli, 16, Milano, 20123, omitted

(21) Appl. No.: 11/883,839

(22) PCT Filed: Feb. 9, 2006
(86) PCT No.: PCT/IB2006/000250

§ 371 Date: Feb. 28, 2008

(30) Foreign Application Priority Data
Feb. 10, 2005 (IT) MO2005A000032

(51) Int. Cl. A61B 17/062

(52) U.S. Cl. 606/148

(57) ABSTRACT

A device (1) for sewing elements that can be sutured, comprising needle means (3), which are shaped so as to form a first arc of a circle and are provided with an end (3a) for piercing at least one suturable element and with an end (3b) for drawing a thread (4) and a sewing head (5) which is associated with a seat (6) for the sliding of the needle means, shaped so as to form a second arc of a circle and provided with a first opening (6a) for the exit of the piercing end (3a) and with a second opening (6b) for the entry of the end, and means (7) for actuating the advancement of the needle means (3) along a path which coincides substantially with the circle, each stroke of the needle means being adapted to form a stitch produced with the thread (4).

### TECHNICAL FIELD

The present invention relates to a device for sewing elements that can be sutured, particularly for suturing body tissues such as blood vessels and hollow organs in general and/or for the endoscopic fixation of prostheses, valves or similar biomedical devices to such tissues.

### BACKGROUND ART

Particularly but not exclusively with reference to the medical and surgical field, it is known to perform endovascular procedures to treat aneurysms, particularly located in the abdominal or thoracic part of the aorta.

As it is known, an aneurysm is a confined dilation of an artery, produced as a consequence of a weakening of the arterial wall caused by a trauma or by a pathological alteration, and can lead to the rupture of the vessel in the affected point, with a consequent risk of death by hemorrhage of the patient.

Two types of procedure are known substantially for obviating this danger: the first one provides for resection of the aneurysm and replacement thereof with a synthetic insert, while the second one is performed endoscopically and/or endovascularly and consists in implanting a prosthesis, constituted by a flexible sheath, inside the vessel at the region affected by the aneurysm by means of appropriate catheters inserted along such vessel under radiographic monitoring.

This prosthesis, by adhering to the internal wall of the vessel upstream and downstream of the dilated portion, returns the passage section of the vessel to the original size and replaces the portion of aorta affected by the aneurysm.

The prosthesis is generally made of synthetic material.

In order to facilitate its adhesion to the internal wall of the vessel and its retention of the correct position along the vessel by friction, known as fixation, traditional prostheses are generally provided at their ends with expandable frames known as stents.

Fixation of the proximal end of the prosthesis, i.e., of the end arranged at the blood inflow side, is of course particularly critical.

The endovascular procedure is evidently particularly interesting with respect to traditional surgery, since it can be performed under local anesthetics, reduces greatly the patient hospitalization time, is less invasive, and is consequently subject to reduced collateral complications.

However, this operating procedure is not free from drawbacks, linked in particular to the possibility of the occurrence, over time, of a migration of the prosthesis inserted in the vessel or of a so-called primary or secondary endoleak, i.e., a separation of the prosthesis from the wall of the vessel, with consequent leakage of blood outside the prosthesis and failure to completely exclude the aneurysm, which can continue to expand.

In all these cases, the patients must in fact be subjected to an invasive surgical procedure, considerably extending their recovery times and increasing the health risks due to the typical complications of these operations.

Another drawback of procedures performed endoscopically and/or endovascularly is that they cannot be performed in patients affected by anatomical constraints which make prosthesis fixation inside the vessel absolutely unstable.

For example, if the aneurysm is located in the abdominal part of the aorta arranged below the insertion of the kidney arteries and the patient has the so-called “collar”, i.e., the portion of aorta comprised between said arteries and the aneurysm has a particular shape and size, an endoscopic and/or endovascular procedure cannot be performed and the patient must be treated by traditional surgery.

Moreover, it is noted that the traditional surgical procedure for treating aneurysms entails immediate complications for the patient, which are considerably greater than those due to an endoscopic and/or endovascular procedure.

In order to obviate these drawbacks, it is known to use endoscopic staplers, which after the release and positioning of the prosthesis are inserted along the vessel with the aid of suitable catheters in order to fix, by means of staples, the proximal end of the prosthesis to the wall of the vessel.

However, even the use of these staplers does not allow to solve in an optimum manner the drawbacks noted above of this type of procedure, since the staples, inserted in a substantially straight or otherwise open configuration, must be deformed and closed up in order to achieve fixation of the prosthesis, with considerable stress for the affected tissues.

### DISCLOSURE OF THE INVENTION

The aim of the present invention is to eliminate the above-mentioned drawbacks of the background art, by providing a device for sewing elements that can be sutured, which allows to achieve optimum and durable fixation of prostheses, valves and similar biomedical devices within blood vessels or hollow organs in general, without producing any stress of the involved tissues.

Within this aim, an object of the present invention is to extend the indication of endoscopic and/or endovascular procedures also to patients with particular anatomical variations or limitations

Another object of the present invention is to allow the provision of seams on suturable elements of any kind, particularly on body tissues, by acting from a single side thereof, without requiring access to the opposite side thereof.

Another object of the present invention is to provide a device which is simple, relatively easy to provide in practice, safe in use, effective in operation, and has a relatively low cost.

This aim and these and other objects, which will become better apparent hereinafter, are achieved by the present device for sewing elements that can be sutured, characterized in that it comprises needle means, which are shaped so as to form a first arc of a circle and are provided with an end for piercing at least one suturable element and with an end for drawing a thread, and a sewing head which is associated with a seat for the sliding of the needle means, shaped so as to form a second arc of said circle and provided with a first opening for the exit of the piercing end and with a second opening for the entry of said end, and with means for actuating the advancement of the needle means along a path which coincides substantially with said circle, each stroke of the needle means being adapted to form a stitch produced with the thread.

### BRIEF DESCRIPTION OF THE DRAWINGS

Further characteristics and advantages of the present invention will become better apparent from the following detailed description of a preferred but not exclusive embodiment of a device for sewing elements that can be sutured, illustrated by way of non-limiting example in the accompanying drawings, wherein:

FIGS. 1 and 2 are two schematic sectional side views of the sewing head of the device according to the invention during work;

FIGS. 3 and 4 are two schematic perspective views of the sewing head during work, which correspond respectively to FIGS. 1 and 2 and with the suturable elements shown in phantom lines;

FIG. 5 is a schematic sectional side view of the sewing head of the device according to the invention, arranged adjacent to the suturable element;

FIG. 6 is a schematic sectional side view of the sewing head of the device according to the invention, returned to a longitudinal position with respect to the elements to be sutured;

FIGS. 7, 8 and 9 are schematic sectional front views of the sewing head of the device according to the invention, illustrating respectively the actuation means, the application means, and the pick-up means;

FIG. 10 is a schematic sectional side view of the device according to the invention during insertion along the elements to be sutured;

FIGS. 11 and 12 are two schematic sectional side views of the device according to the invention during work;

FIGS. 13 and 14 are two schematic transverse sectional views of the guiding means of FIG. 12, with the reference means respectively deactivated and activated;

FIG. 15 is a schematic transverse sectional view of the sewing head of FIG. 12, with the temporary positioning means activated.

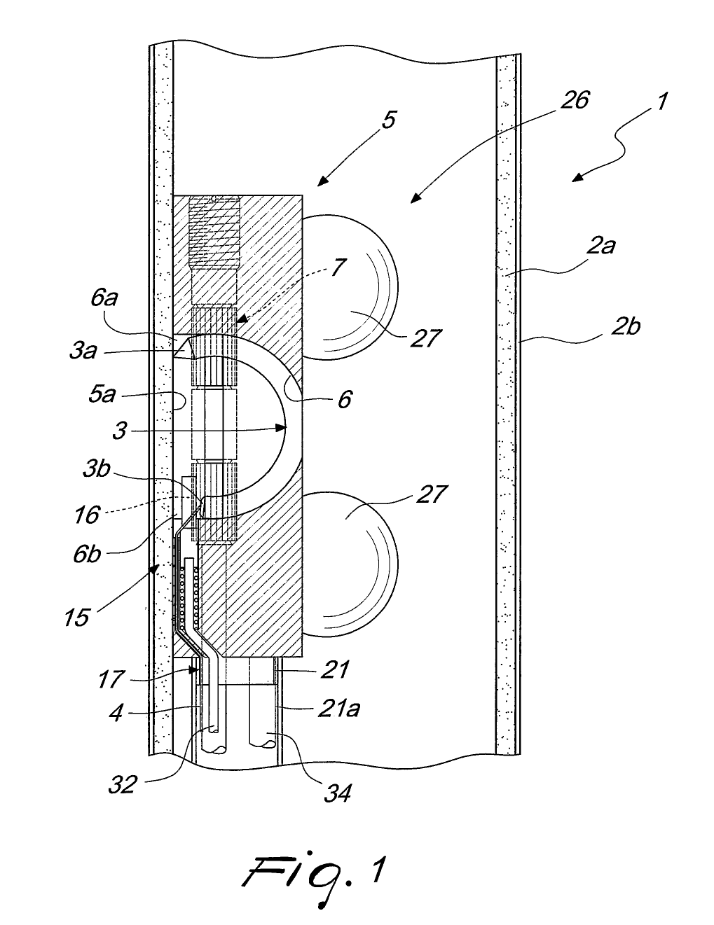

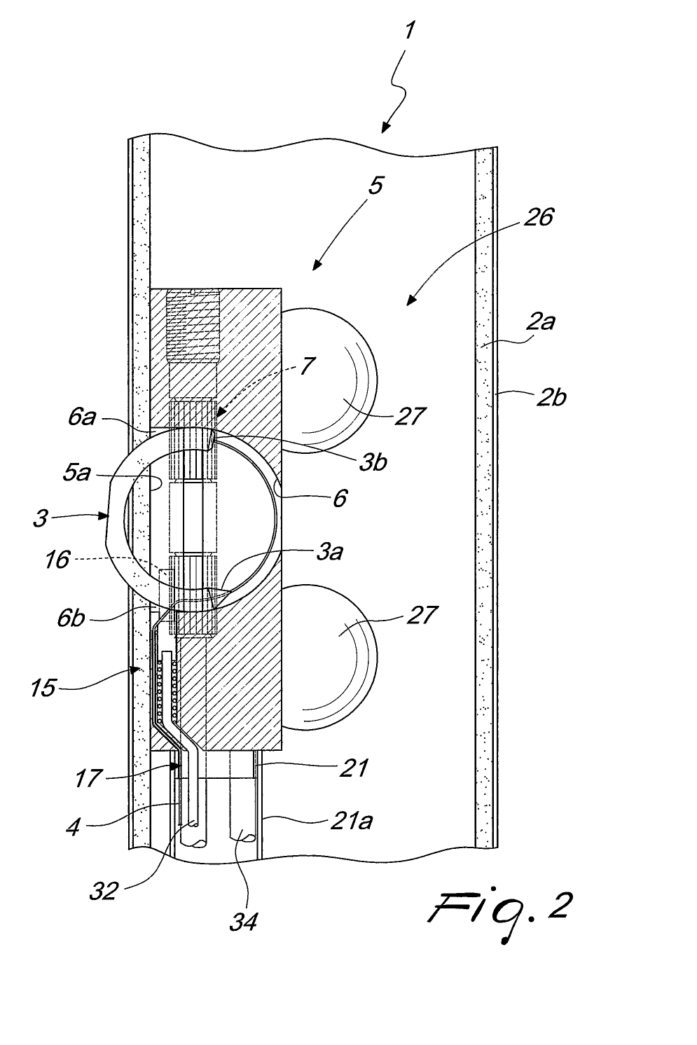

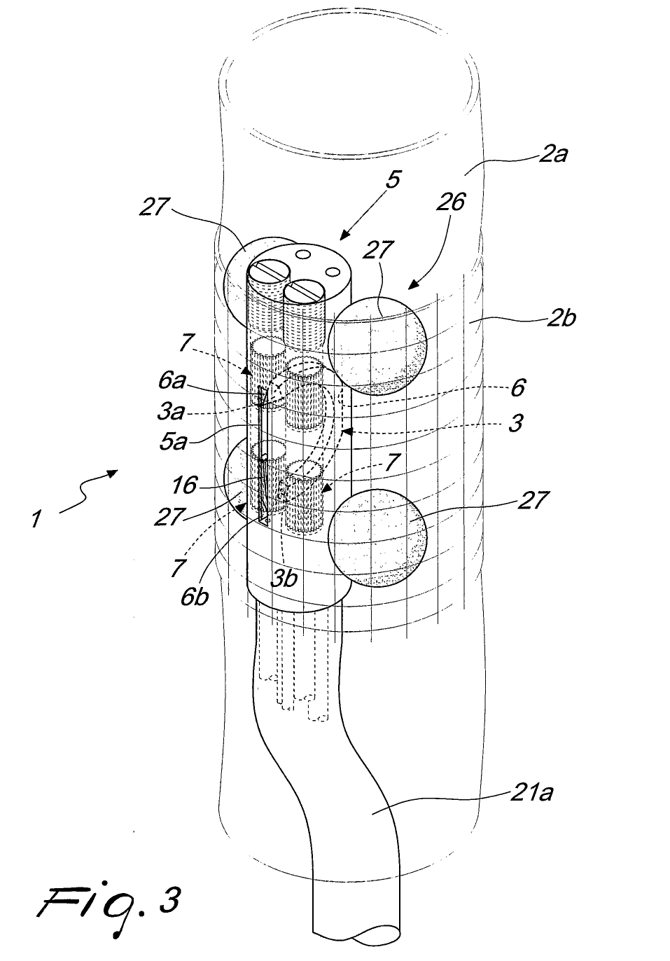

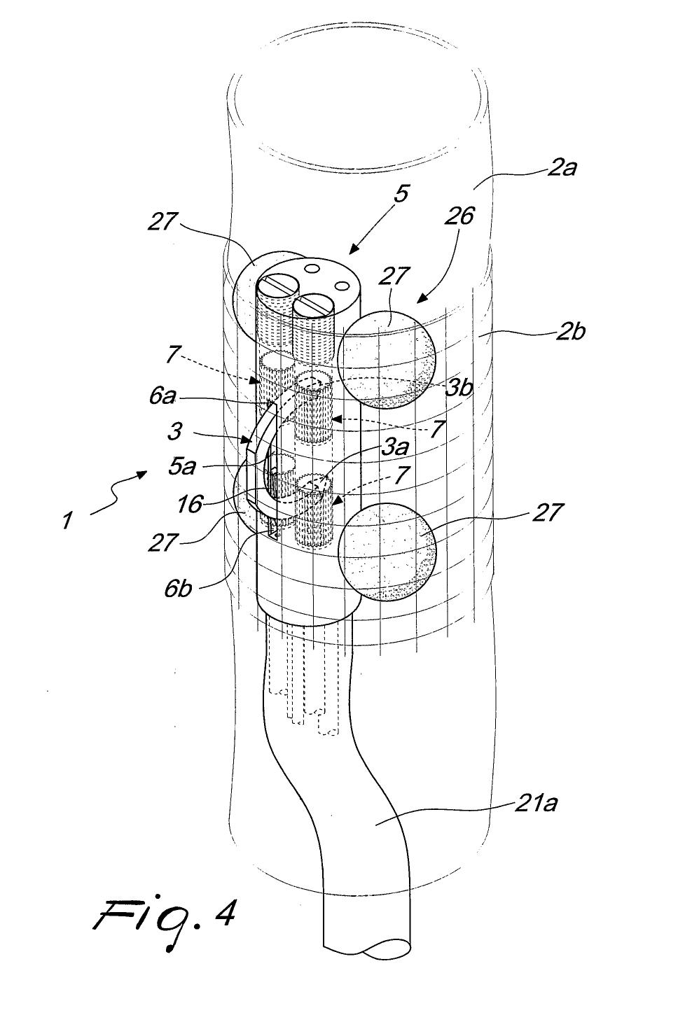

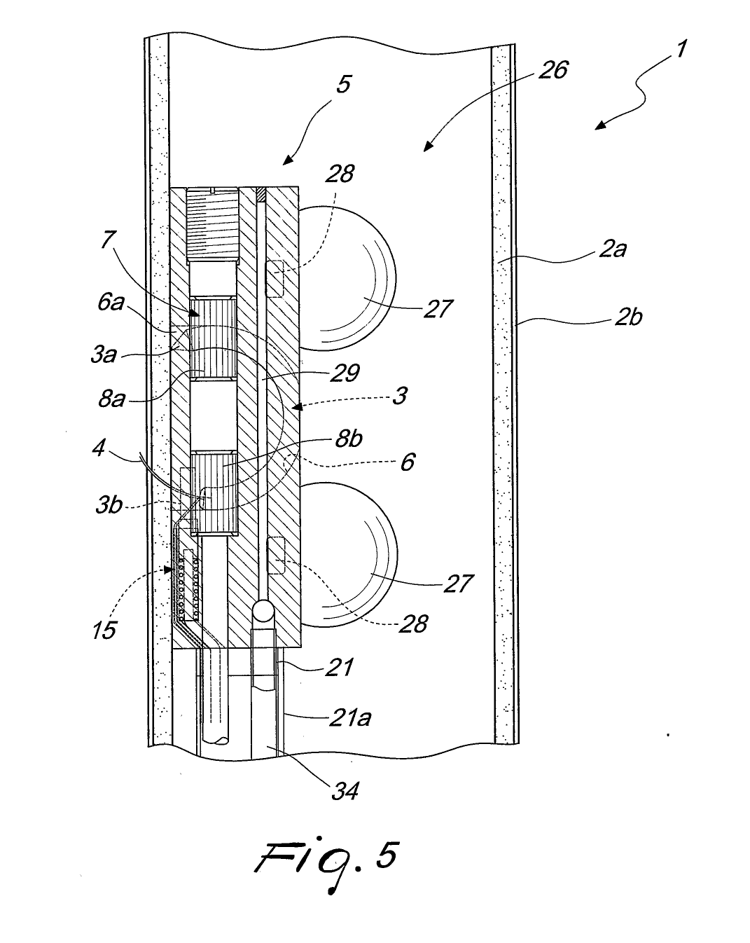

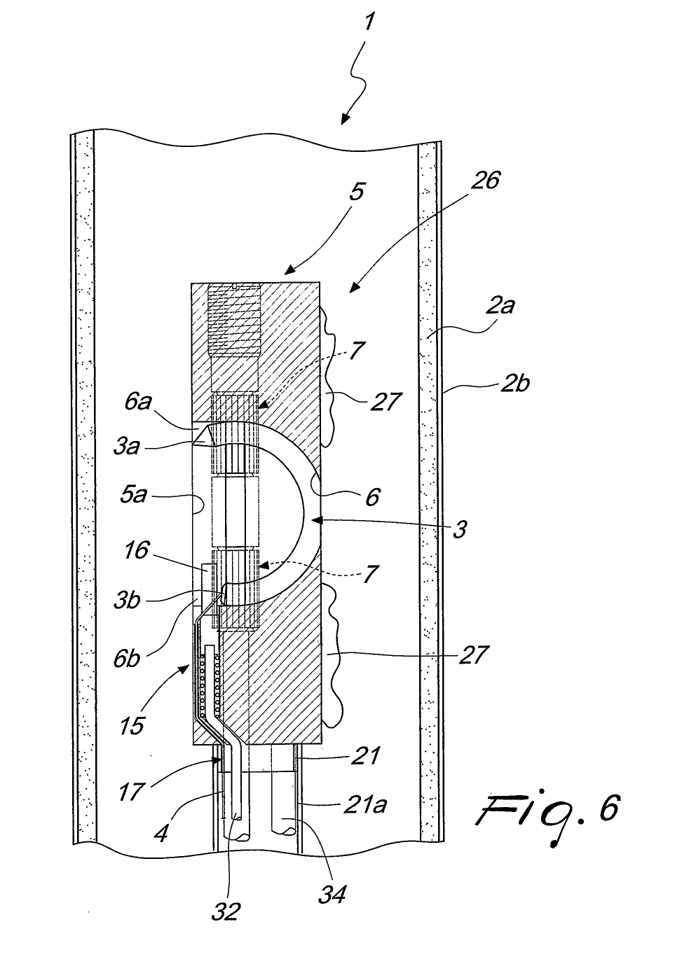

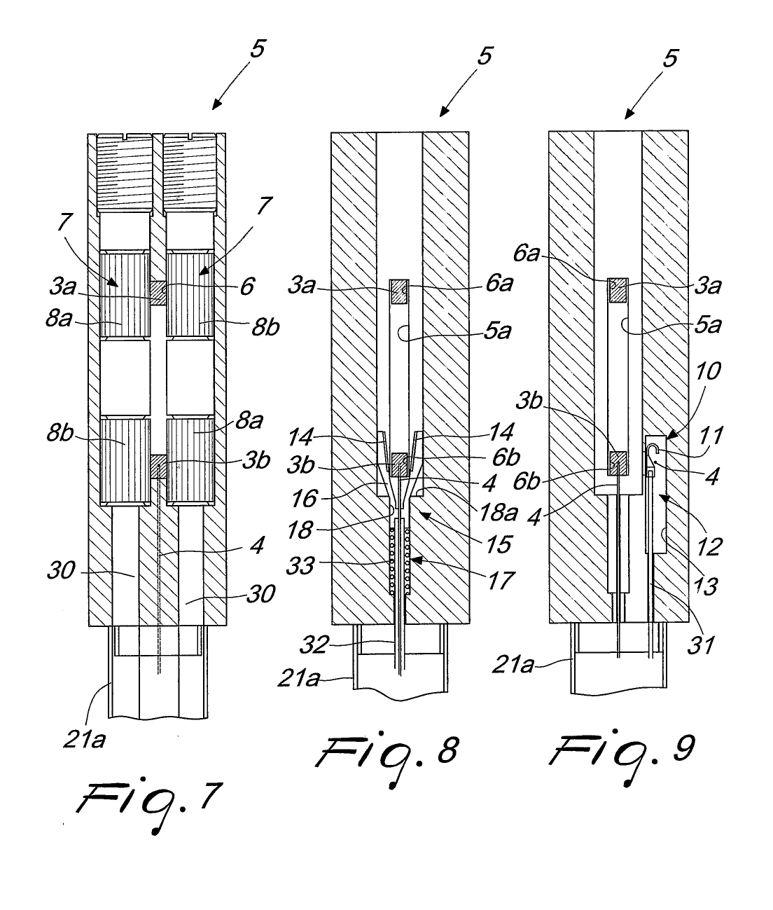

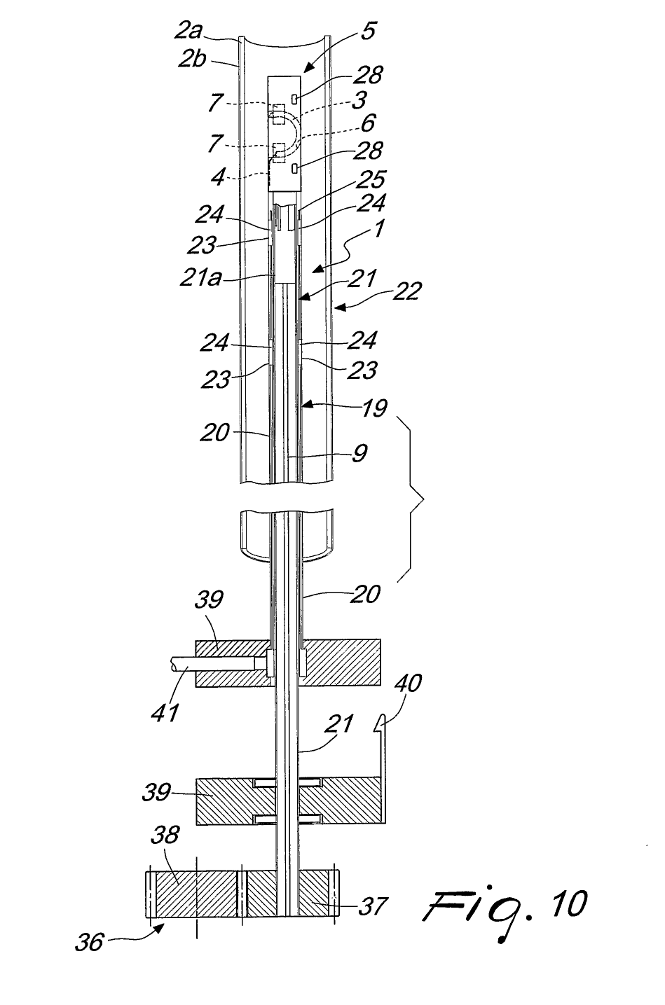

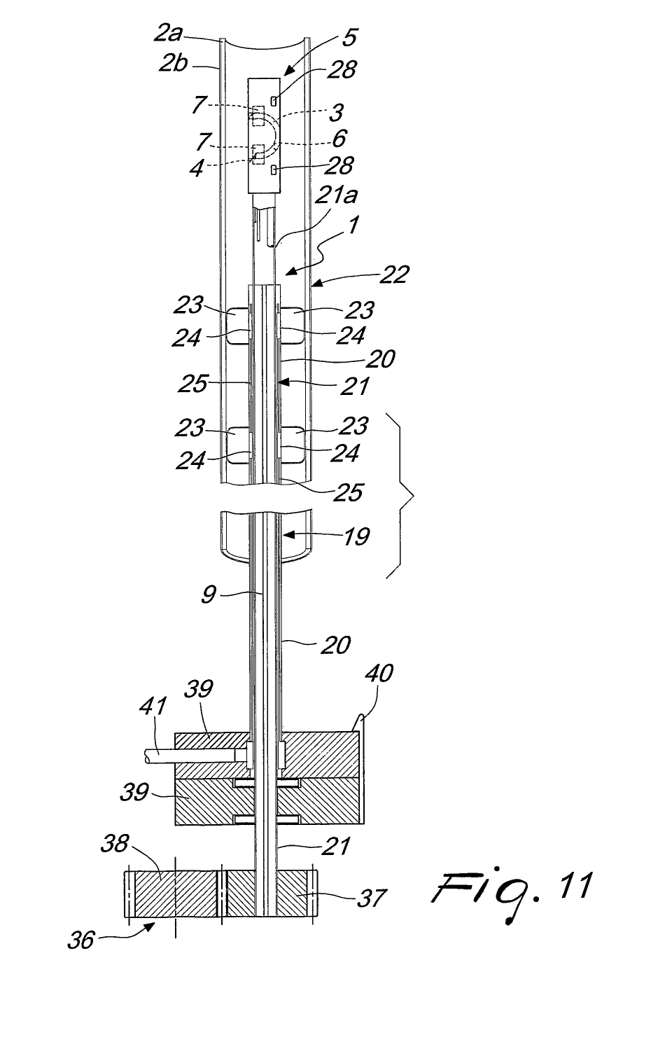

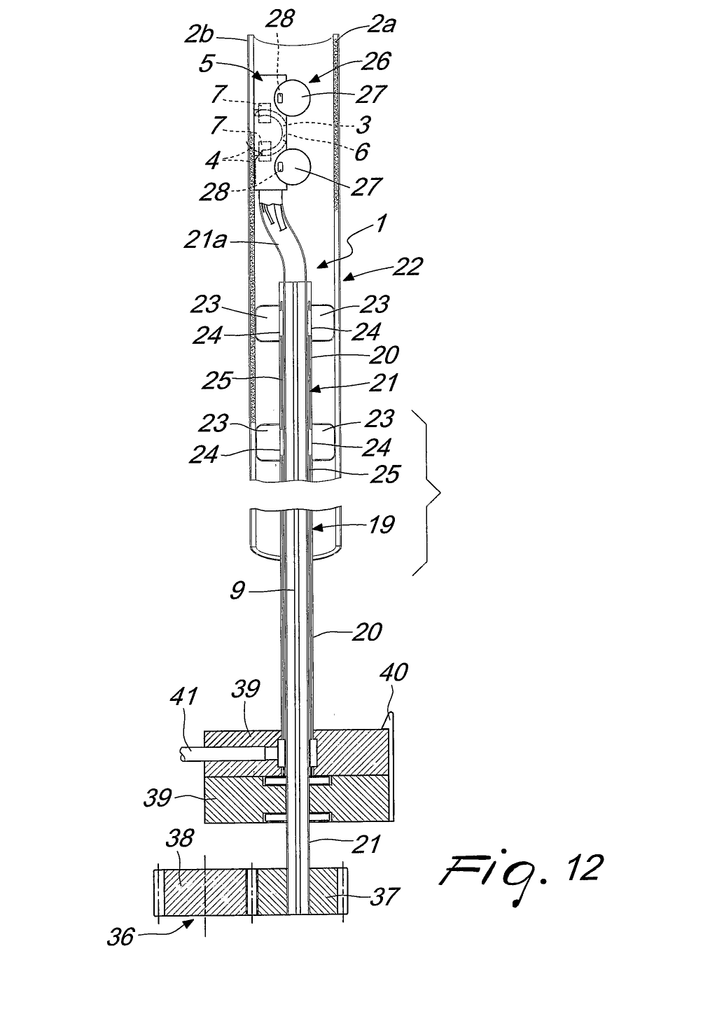

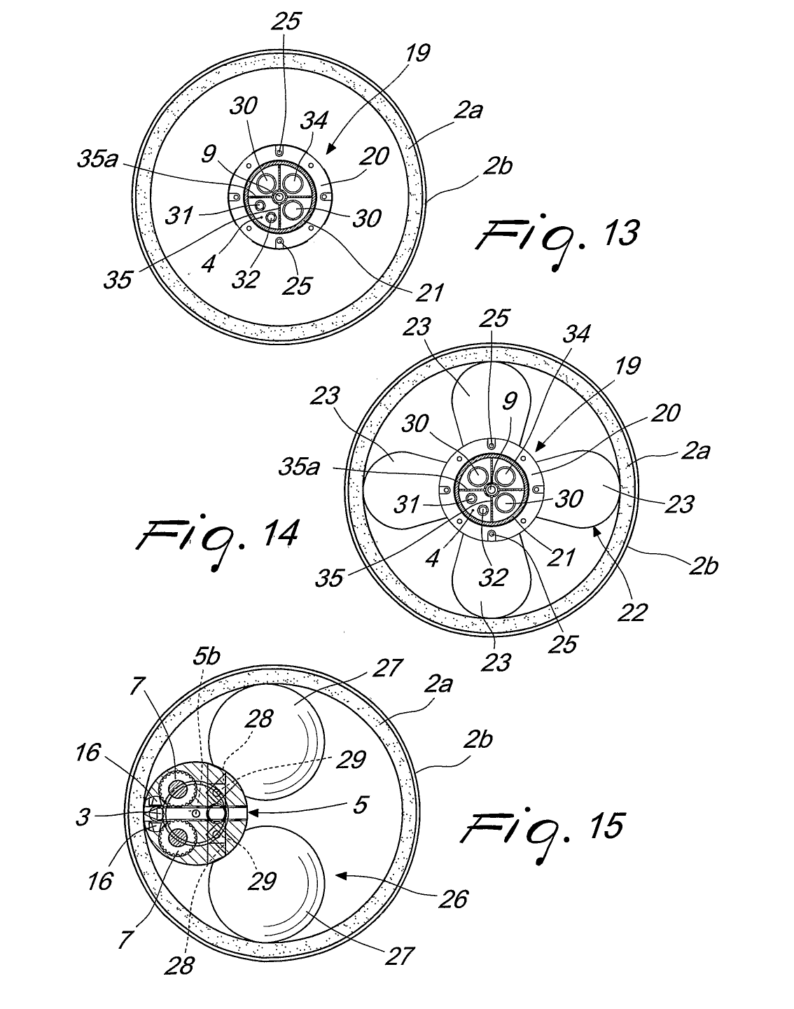

### WAYS OF CARRYING OUT THE INVENTION

With reference to the figures, the reference numeral 1 generally designates a device for sewing elements 2 that can be sutured, particularly for suturing body tissues such as blood vessels and hollow organs in general and/or for the endoscopic and/or endovascular fixation of prostheses, valves or similar biomedical devices to such tissues.

The device 1 can be used both to join two or more suturable elements 2 which are mutually superimposed and to join the lips of a wound, cut, laceration or the like that has occurred on a suturable element 2.

In the medical-surgical field, for example, the device can be applied to suture wounds or cuts from the outside, ensuring the execution of sutures which are not excessively penalizing from the aesthetic standpoint since they are constituted by uniform stitches, or to perform endosutures to heal cuts or lacerations of blood vessels or hollow organs from the inside thereof or for the fixation of prostheses, valves or the like.

The device 1 can be applied to perform linear seams and seams closed in a loop on suturable elements 2 which have a tubular shape or the like.

Moreover, the device 1 can be used by acting from a single side of the suturable element 2 and without requiring direct access also to the opposite side with respect to the one from which one is operating.

The device 1 comprises needle means 3, which are shaped so as to form a first arc of a circle and are provided with an end 3a for piercing one or more mutually superimposed suturable elements 2 and with an end 3b for drawing a thread 4.

The device 1 further comprises a sewing head 5, which is associated with a seat 6 for the sliding of the needle means 3, which is contoured so as to form a second arc of said circle and is provided with a first opening 6a for the exit of the piercing end 3a and with a second opening 6b for the entry of said end, and with means 7 for actuating the advancement of the needle means 3 along said seat, along a path which substantially coincides with said circle.

The openings 6a and 6b can be formed by the end portions of a single longitudinal slot 5a provided in the sewing head 5.

Each stroke of the needle means 3, which corresponds to a rotation through 360° along said circle, forms a stitch produced by means of the thread 4.

Advantageously, the center angle subtended by the first arc is substantially greater than the complementary angle to 360° of the center angle subtended by the second arc, so as to ensure that the needle means 3 are always engaged along the seat 6 in their advancement motion.

The seat 6 is preferably provided so that it passes through the sewing head 5.

The actuation means 7 comprise at least one pair of contrarotating rollers 8, of which at least one 8a is motorized so as to turn about its own longitudinal axis and the other roller 8b is idle, said rollers being supported by the sewing head 5 on opposite sides of the needle means 3 and being arranged so that their corresponding lateral surfaces are in contact therewith.

The rotation of the rollers 8 is adapted to transmit the advancement motion to the needle means 3 by friction; in order to increase the friction coefficient between the rollers 8 and the needle means 3, the lateral surfaces of said rollers are provided rough, for example by knurling.

Preferably, the rollers 8 are accommodated within the sewing head 5.

In the illustrated embodiment there are two pairs of rollers 8 arranged along the seat 6, each proximate to a corresponding opening 6a or 6b.

Each pair of rollers 8 is provided with a corresponding motorized roller 8a and with a corresponding idle roller 8b, the motorized roller 8a arranged at the first opening 6a being aligned with the free roller 8b arranged at the second opening 6b and vice versa.

If used for seams on suturable elements 2 which are substantially flat or in any case of seams produced from the outside of said suturable elements, the device 1 is provided with a handle and preferably with a spool for feeding the thread 4.

The figures show, merely by way of example, the application of the device 1 for sewing two suturable elements 2 which are substantially tubular and are arranged one inside the other and in particular for fixing a prosthesis 2a of the type commonly implanted to treat aneurysms on the internal wall of a blood vessel 2b.

For endoscopic and/or endovascular use, according to a conventional operating method, the device 1 is inserted along the vessel 2b until it reaches the point where the prosthesis 2a is to be fixed under radiographic monitoring, and is fitted on a guiding catheter 9 previously inserted along said vessel in order to implant the prosthesis 2a.

For this purpose, the sewing head 5 is provided with a longitudinal through hole 5b for fitting on the guiding catheter 9.

The sewing head 5 is substantially cylindrical and elongated in the longitudinal direction of the suturable elements 2.

The seat 6 is provided on a substantially diametrical plane of the sewing head 5 and its openings 6a and 6b are aligned along their longitudinal direction.

The device 1 comprises means 10 for picking up the thread 4 after forming each stitch in order to perform the next stitch.

It should be noted that the pick-up means 10 are arranged laterally with respect to the seat 6, so that they engage the portion of thread 4 which is comprised between the stitch that has just been formed and the drawing end 3b.

The pick-up means 10 comprise at least one hook-shaped element 11, which can engage the thread 4 and is associated slidingly with the sewing head 5, and means 12 for the alternating movement of said hook-shaped element between two end configurations.

Along the forward stroke between said end configurations, the hook-shaped end 11 engages the thread 4 and draws it through the stitch or stitches that have already been formed, drawing a portion thereof to form the next stitch.

The hook-shaped element 11 is preferably accommodated within the sewing head 5.

A channel 13 is formed within the sewing head 5, is aligned with the longitudinal direction of said head and runs laterally with respect to the seat 6, and at the second opening 6b is extended away from the first opening 6a.

In the inactive configuration, the hook-shaped element 11 is arranged at the second opening 6b, and slides along the channel 13 when the movement means 12 are actuated.

If the seam to be provided is closed in a loop, the device 1 also has means for coupling the portions of thread 4 which protrude from the first and last stitches formed and preferably has means for cutting said excess portions of thread 4.

In the illustrated embodiment, said coupling means and said cutting means are mutually monolithic and comprise two plates 14, which can be mated together by interlocking and are provided with respective sharp edges.

The ends of the portions of thread 4 which protrude from the first and last stitches that are formed are accommodated and locked between the plates 14 and cleaned of the excess thread 4 by the sharp edges of said plates.

The device 1 comprises means 15 for applying the plates 14, which are associated with the sewing head 5.

The application means 15 comprise an elastic clamp 16, which is provided with two supporting claws for respective plates 14 and is movably associated with the sewing head 5 and with means 17 for actuating the closure and opening of said claws.

The clamp 16 is preferably accommodated within the sewing head 5.

The clamp 16 is arranged so that the corresponding claws close at the second opening 6b, so as to affect the portions of thread 4 which protrude from the first and last stitches formed at their point of intersection.

The actuation means 17 comprise an elongated receptacle 18 for the alternating sliding of the clamp 16, which is shaped so as to form a narrower cross-section 18a, is aligned with the longitudinal direction of the sewing head 5, and intersects the seat 6 proximate to the second opening 6b.

In the inactive configuration, the clamp 16 is arranged so that the corresponding claws are divaricated on opposite sides of the seat 6, while by actuating the actuation means 17 it slides along the receptacle 18.

In the forward stroke through the narrower cross-section 18a, the claws of the clamp 16 close, ensuring the coupling of the plates 14.

For the provision of seams on suturable elements 2 which have a tubular shape and particularly for endoscopic use, the device 1 comprises guiding means 19 for inserting the sewing head 5 along said elements.

The sewing head 5 must be arranged along the suturable elements 2 so that the seat 6 lies on a plane which is substantially perpendicular to said elements and so that the openings 6a and 6b face them.

The guiding means 19 comprise a first external supporting cannula 20, and a second cannula 21, which supports the sewing head 5 and is inserted substantially snugly within the first cannula 20.

The cannulas 20 and 21 are made of flexible material of the type used conventionally to manufacture catheters for endoscopic and/or endovascular use.

The cannulas 20 and 21 are provided with corresponding first and second ends, which are arranged respectively inside and outside the suturable elements 2.

In the case of endoscopic and/or endovascular use, in particular, the first ends of the cannulas 20 and 21 are arranged outside the human body, and said cannulas are inserted along the vessel 2b until they reach the prosthesis 2a through an incision formed for example at the groin; the second ends of the cannulas 20 and 21 are instead kept outside the human body for the actuation of the operation of the device 1 by an operator.

The first cannula 20 and the second cannula 21 preferably can slide and/or rotate with respect to each other.

In particular, it is possible to actuate the controlled sliding of the first cannula 20 with respect to the second cannula and the relative rotation of the second cannula 21 with respect to the first one.

The sewing head 5 is rigidly associated with the first end of the second cannula 21.

During insertion along the vessel 2b, the second cannula 21 is kept inside the first cannula 20, so that the sewing head 5 protrudes from the first end of the first cannula 20; once the work area has been reached, the first cannula 20 is extracted partially so as to expose the end portion 21a of the second cannula 21.

The thread 4 passes through the second cannula 21.

Conveniently, the device 1 is provided with reference means 22 for the positioning of the first cannula 20 with respect to the suturable elements 2, so as to ensure alignment and concentricity with respect to them.

The reference means 22 have a first plurality of expandable elements, which are distributed along the peripheral region of the first cannula 20 and are adapted to abut against the internal wall of the prosthesis 2a and/or of the vessel 2b.

Said expandable elements are constituted by first bags 23, which when inactive are accommodated within respective first recesses 24 formed on the outer surface of the first cannula 20 and are associated with respective first ducts 25 for feeding a pressurized fluid, which are formed along said first cannula.

In the illustrated embodiment there are two assemblies, each constituted by four first bags 23 which are arranged along the peripheral region of the first cannula 20 and are distributed longitudinally along said first cannula proximate to the corresponding first end.

The device 1 further comprises means 26 for temporarily positioning the sewing head 5 so that the openings 6a and 6b are substantially adjacent to the internal wall of the prosthesis 2a during work.

The positioning means 26 have a second plurality of expandable elements, which are distributed along the peripheral region of the sewing head 5 and are adapted to abut against the internal wall of the prosthesis 2a.

In particular, said expandable elements are arranged on the opposite side of the sewing head 5 with respect to the openings 6a and 6b.

Said expandable elements are constituted by second bags 27, which when inactive are accommodated within respective second recesses 28 formed on the external surface of the sewing head 5 and are associated with respective second ducts 29 for feeding a pressurized fluid, which are provided along said sewing head.

In the illustrated embodiment there are two assemblies, each constituted by two second bags 27 arranged on the peripheral region of the sewing head 5 and distributed longitudinally along said sewing head.

The device 1 can conveniently have means for adjusting the relative arrangement of the openings 6a and 6b with respect to the suturable elements 2, not shown, so as to adapt the distance at which said openings are kept during work by the positioning means 26 with respect to the wall of the suturable element 2 which faces them, depending on the overall thickness that the needle means 3 must pierce and so as to compensate, for an equal stroke of the needle means 3 with respect to the sewing head 5, a variation of said thickness.

For use of the device 1 in the endovascular field, said adjustment means allow to compensate variations in the thickness of the vessel 2b caused by calcification of its internal wall, optionally revealed by radiographic monitoring.

The adjustment means can have a third plurality of expandable elements distributed on the peripheral region of the sewing head 5 proximate to the openings 6a and 6b and adapted to abut against the internal wall of the prosthesis 2a.

Said expandable elements also can be constituted by third bags, which are accommodated, when inactive, within respective third recesses provided on the external surface of the sewing head 5 and are associated with respective third ducts for feeding a pressurized fluid, which are formed along said sewing head.

The device 1 comprises at least one flexible cable 30 for transmitting rotation to the at least one motorized roller 8a, which is accommodated along the second cannula 20; in the illustrated embodiment there are two flexible cables 30, each of which has an end which is associated with a respective motorized roller 8a and an opposite end which protrudes from the cannulas 20 and 21 and is coupled to conventional motor means, not shown.

The movement means 12 comprise a first traction element 31, which is associated with the hook-shaped element 11, passes through the channel 13 and is accommodated along the second cannula 21, for actuating the sliding of said hook-shaped element at least along the forward stroke.

The first traction element 31 has an end which is associated with the hook-shaped element 11 and an opposite end which protrudes from the cannulas 20 and 21 and is associated with conventional means for grip and actuation on the part of an operator, which are not shown.

The movement means 12 may also have a first elastic element, not shown, which is interposed between the hook-shaped element 11 and the sewing head 5 at the channel 13 in order to actuate the sliding of said hook-shaped element along the return stroke.

The actuation means 17 comprise a second traction element 32, which is associated with the clamp 16, passes through the receptacle 18 and is accommodated along the second cannula 21, for the actuation of the sliding of said clamp along at least the forward stroke.

The second traction element 32 has an end which is associated with the clamp 16 and an opposite end which protrudes from the cannulas 20 and 21 and is associated with conventional means, not shown, for grip and actuation on the part of an operator.

The actuation means 17 further have a second elastic element, which is interposed between the clamp 16 and the sewing head 5 at the receptacle 18 for actuating the sliding of said clamp along the return stroke.

The second elastic element is constituted by a helical compression spring 33, which is wound around the second traction element 32 and is interposed between the clamp 16 and the bottom of the receptacle 18.

The positioning means 26 comprise at least one channel 34 for feeding the pressurized fluid toward the second ducts 29, which passes along the second cannula 21.

The device 1 has a longitudinal core 35, which is accommodated along the second cannula 21 and is shaped so as to divide its transverse cross-section into a plurality of sectors, particularly four sectors, of which a first sector and a second sector each accommodates a respective flexible cable 30, a third sector accommodates the feeder channel 34, and a fourth sector accommodates the first traction element 31, the second traction element 32 and the thread 4.

The core 35 is provided with a through hole 35a at the central portion for fitting on the guiding catheter 9.

Preferably, the core 35 is interrupted at the end portion 21a, which must be more flexible in order to facilitate the arrangement of the sewing head adjacent to the suturable elements 2.

The device 1 comprises means 36 for turning the sewing head about its own longitudinal axis which are associated with the second cannula 21 and comprise a first gear 37, which is keyed rigidly to said second cannula proximate to the corresponding second end, and a second motorized gear 38 which meshes with said first gear.

The rotation means 36 are of the intermittent type, since they must allow successive rotations equal to a preset angle of the second cannula 21 with respect to the first cannula 20 and therefore of the sewing head 5 with respect to the elements 2 to be sutured.

Finally, the device 1 is provided with respective supporting blocks 39, which can be mutually coupled by interlocking, for example by means of a hook 40, and are associated with the cannulas 20 and 21 at the corresponding second ends.

The distance between the blocks 39 during insertion of the device 1 substantially matches the length of the end portion 21a which protrudes from the first cannula 20 during work, following the coupling of the blocks 39.

The block 39 associated with the first cannula 20 is coupled to conventional means 41 for feeding the pressurized fluid along the first ducts 25, which are not shown in detail.

It should be noted that by way of the reference means 22, the longitudinal positioning of the first cannula 20 with respect to the suturable elements 2 is ensured, while the coupling of the blocks 39 ensures the longitudinal positioning of the entire device 1 during work.

The operation of the device according to the invention is substantially as follows.

The device 1 is inserted along the suturable elements 2 so as to position the sewing head 5 so that the openings 6a and 6b face the region where the prosthesis 2a is to be sewn to the vessel 2b.

The first cannula 20 is made to slide over the second cannula 21 until the two blocks 39 are mated and then the reference means 22 are actuated so that the first bags 23 inflate.

To perform each stitch, the positioning means 26 are actuated so as to move the sewing head 5 so that its openings 6a and 6b are adjacent to the internal wall of the prosthesis 2a, the motorized rollers 8a are turned until the needle means 3 perform a circular stroke, and then the second bags 27 are deflated at least partially in order to move the sewing head 5 away from said prosthesis.

Following the forming of each stitch, the pick-up means 10 are actuated in order to pick up a portion of thread 4, and by way of the rotation means 36, the second cannula 21 is turned with respect to the first cannula 20 through a preset angle so as to arrange the sewing head 5 in order to form the next stitch.

The application means 15 are actuated at the end of the sewing process so as to release the plates 14 as described above.

Substantially, the sewing method according to the invention consists in positioning needle means, shaped so as to form an arc of a circle and provided with an end for piercing at least one suturable element and with an end for drawing a thread in an initial configuration in which they are substantially perpendicular to a first face of the suturable element and with the piercing end proximate to said first face, and in actuating the advancement of the needle means along a path which coincides substantially with said circle in the direction in which the piercing end moves toward the first face in order to achieve a first crossing of the thickness of the suturable element from the first face toward the second face, which lies opposite the first one, a subsequent second crossing of the thickness of the suturable element from the second face toward the first face, and the return of the needle means to the initial configuration so as to form a stitch.

In order to sew several suturable elements which are mutually superimposed, the needle means are made to advance so as to achieve a first crossing of the thickness of said suturable elements starting from said first face, a subsequent second crossing of the thickness of said suturable elements in the opposite direction with respect to the first crossing, and the return of the needle means to the initial configuration so as to form a stitch.

If it is necessary to provide several stitches, the method according to the invention provides for a step for the translational motion of the needle means by a preset extent along a direction which coincides substantially with the plane of arrangement of the stitch.

To produce a plurality of stitches and obtain the seam, it is necessary to repeat several times the steps for activating the advancement and translational motion of the needle means.

In practice it has been found that the described invention achieves the intended aim and objects.

In particular, the device according to the invention allows to produce endosutures on body tissues which ensure optimum fixation of prostheses or the like without causing any stress or damage to said tissues.

More generally, the device according to the invention allows to provide linear seams and seams closed in a loop on suturable elements having any shape, whether flat, tubular or otherwise shaped, without requiring access to both sides of said suturable elements.

The invention thus conceived is susceptible of numerous modifications and variations, all of which are within the scope of the appended claims.

All the details may further be replaced with other technically equivalent ones.

In practice, the materials used, as well as the shapes and the dimensions, may be any according to requirements without thereby abandoning the scope of the protection of the appended claims.

The disclosures in Italian Patent Application No. MO2005A000032, from which this application claims priority, are incorporated herein by reference.

What is claimed is:

1-44. (canceled)

45. A device for sewing elements that can be sutured, comprising needle means, which are shaped so as to form a first arc of a circle and are provided with an end for piercing at least one suturable element and with an end for drawing a thread and a sewing head which is associated with a seat for the sliding of said needle means, shaped so as to form a second arc of said circle and provided with a first opening for the exit of said piercing end and with a second opening for the entry of said end, and means for actuating the advancement of said needle means along a path which coincides substantially with said circle, each stroke of said needle means being adapted to form a stitch produced with said thread.

46. The device of claim 45, wherein the center angle subtended by said first arc is substantially greater than a complementary angle to 360° of a center angle subtended by said second arc.

47. The device of claim 45, wherein said seat is formed through the inside of said sewing head.

48. The device of claim 45, wherein said actuation means comprise at least one pair of contrarotating rollers, at least one of which is motorized so as to turn about its own longitudinal axis, said rollers being supported by said sewing head on opposite sides of said needle means and being arranged so that their corresponding lateral surfaces are in contact with them, the rotation of the rollers being adapted to transmit by friction the advancement motion to the needle means.

49. The device of claim 45, wherein said actuation means are accommodated inside said sewing head.

50. The device of claim 45, further comprising means for picking up said thread following the production of each stitch, in order to produce the next stitch.

51. The device of claim 50, wherein said pick-up means comprise at least one hook-shaped element, which can engage said thread and is associated slidingly with said sewing head, and means for the alternating movement of said hook-shaped element between two end configurations, the hook-shaped element being adapted, along the forward stroke, to engage the thread and to draw a portion thereof in order to form the next stitch.

52. The device of claim 51, wherein said hook-shaped element is accommodated inside said sewing head.

53. The device of claim 50, further comprising means for retaining portions of thread which protrude from first and last stitches formed, a seam being closed in a loop.

54. The device of claim 53, further comprising means for cutting said portions of thread which protrude from the first and last stitches performed by the excess thread.

55. The device of claim 54, wherein said retention means and said cutting means are provided so that they are mutually monolithic and comprise two plates, which can be mutually coupled by interlocking and are provided with respective cutting edges, the end portions of said portions of thread which protrude from the first and last stitches formed being adapted to be accommodated between said plates.

56. The device of claim 55, further comprising means for applying said plates which are associated with said sewing head.

57. The device of claim 56, wherein said application means comprise an elastic clamp, which is provided with two claws for supporting respective plates and is movably associated with said sewing head, and means for actuating the closure and opening of said claws, the claws being adapted to close at said seat and proximate to said second opening.

58. The device of claim 57, wherein said clamp is accommodated inside said sewing head.

59. The device of claim 57, wherein said actuation means comprise a receptacle for the alternated sliding of said clamp, which is contoured so as to form a narrower cross-section, the claws being adapted to close, in order to couple the plates, in the forward stroke of the clamp through the narrower cross-section.

60. The device of claim 48, further comprising guiding means for introducing said sewing head along said suturable element, the suturable element being substantially tubular and the sewing head being adapted to be arranged so that the seat lies on a plane which is substantially perpendicular to said element and so that the first and second openings face it.

61. The device of claim 60, wherein said guiding means comprise a first supporting cannula and a second cannula which supports said sewing head and is inserted substantially snugly within said first cannula, the cannulas being provided with corresponding first and second ends which are adapted to be arranged respectively inside and outside said suturable element.

62. The device of claim 61, wherein said first and second cannulas can rotate and/or slide with respect to each other.

63. The device of claim 45, wherein said sewing head comprises a longitudinal through hole for fitting on a guiding catheter arranged along said suturable element.

64. The device of claim 61, wherein said sewing head is associated with the first end of said second cannula.

65. The device of claim 61, further comprising reference means for the positioning of said first cannula with respect to said suturable element.

66. The device of claim 65, wherein said reference means comprise at least one first plurality of expandable elements, which are distributed on the peripheral region of said first cannula and are adapted to abut against said suturable element.

67. The device of claim 66, wherein said expandable elements are constituted by first bags, which when inactive are accommodated within respective first recesses formed on the external surface of said first cannula and are associated with respective first ducts for feeding a pressurized fluid, which are formed along said first cannula.

68. The device of claim 45, further comprising means for temporarily positioning said sewing head so that said first and second openings are substantially adjacent to said suturable element during work.

69. The device of claim 68, wherein said temporary positioning means comprise a second plurality of expandable elements which are associated with the peripheral region of said sewing head.

70. The device of claim 69, wherein said expandable elements are constituted by second bags, which when inactive are accommodated within respective second recesses formed on the outer surface of said sewing head and are associated with respective second ducts for feeding a pressurized fluid which are formed along said sewing head.

71. The device of claim 45, further comprising means for adjusting the relative position of said first and second openings with respect to said suturable element during work.

72. The device of claim 71, wherein said adjustment means comprise a third plurality of expandable elements which are associated with the peripheral region of said sewing head proximate to said first and second openings.

73. The device of claim 72, wherein said expandable elements are constituted by third bags, which when inactive are accommodated within respective third recesses formed on the outer surface of said sewing head and are associated with respective third ducts for feeding a pressurized fluid, which are formed along said sewing head.

74. The device of claim 61, further comprising at least one flexible cable for transmitting the rotation to said motorized roller, which is accommodated along said second cannula.

75. The device of claim 74, wherein said movement means comprise a first traction element, which is associated with said hook-shaped element and is accommodated along said second cannula in order to actuate the sliding of said hook-shaped element at least along the forward stroke.

76. The device of claim 75, wherein said movement means comprise a first elastic element, which is interposed between said hook-shaped element and said sewing head in order to actuate the sliding of said hook-shaped element along the return stroke.

77. The device of claim 76, wherein said actuation means comprise a second traction element, which is associated with said clamp and is accommodated along said second cannula in order to actuate the sliding of said clamp at least along the forward stroke.

78. The device of claim 77, wherein said actuation means comprise a second elastic element, which is interposed between said clamp and said sewing head in order to actuate the sliding of said clamp along the return stroke.

79. The device of claim 61, wherein said thread passes through and along said second cannula.

80. The device of claim 68, wherein said temporary positioning means comprise at least one channel for feeding pressurized fluid toward said second ducts, which passes through and along said second cannula.

81. The device of claim 61, further comprising a core which is accommodated along said second cannula and is shaped so as to divide an internal section thereof into a plurality of sectors.

82. The device of claim 81, wherein said core is perforated at a central portion for fitting on said guiding catheter.

83. The device of claim 61, further comprising means for turning said head about its longitudinal axis, said means being associated with a first end of said second cannula, said second cannula being rigidly coupled to said sewing head.

84. The device of claim 61, further comprising respective supporting blocks which can be mutually coupled by interlocking during work and are associated with said first and second cannulas proximate to corresponding second ends of said cannulas.

85. The device of claim 45, wherein said first and second openings are formed by end portions of a slot formed in said sewing head.

86. A method for sewing elements that can be sutured, comprising the steps of:
a. positioning needle means, contoured so as to form an arc of a circle and provided with an end for piercing at least one element that can be sutured and with an end for drawing a thread in an initial configuration in which they are arranged substantially at right angles to a first face of said suturable element and with the piercing end proximate to said first face;
b. actuating the advancement of said needle means along a path which coincides substantially with said circle in the direction for moving said piercing closer to said first face in order to achieve a first crossing of the thickness of the suturable element from the first face toward the second face, which lies opposite the first face, a subsequent second crossing of the thickness of the suturable element from the second face toward the first face, and the return of the needle means to the initial configuration so as to form a stitch.

87. The method of claim 86, further comprising the step of:
c. moving said needle means by an extent along a direction which substantially coincides with the arrangement of said seam.

88. The method of claim 87, wherein the succession of said steps b) and c) is repeated several times in order to produce a plurality of stitches and obtain a seam.
